(19) United States
(12) Patent Application Publication
(10) Pub. No.: US 2007/0293433 A1
(43) Pub. Date: Dec. 20, 2007

(54) Methods of treating aging of skin with oligosaccharides in cosmetic or dermatological compositions that stimulate adhesion of keratinocytes to major proteins of the dermoepidermal junction and restore epidermal cohesion

(75) Inventors: Christian Lubrano, Issy-les-Moulineaux (FR); Laurence Flavet, Montigny-le-Bretonneux (FR); Gaelle Saintigny, Paris (FR); Jean-Renaud Robin, Nanterre (FR)

Correspondence Address: IP GROUP OF DLA PIPER US LLP, ONE LIBERTY PLACE, 1650 MARKET ST, SUITE 4900, PHILADELPHIA, PA, 19103, US

(73) Assignee: Laboratoires de Biologie Vegetale Yves Rocher, a corporation of France, Issy-les-Moulineaux (FR)

(21) Appl. No.: 11/894,945

(22) Filed: Aug. 22, 2007

Related U.S. Application Data
(62) Division of application No. 10/839,420, filed on May 5, 2004.
Application No. 10/839,420 is a continuation of application No. PCT/FR02/03844, filed on Nov. 8, 2002.

(30) Foreign Application Priority Data
Nov. 8, 2001 (FR) FR 01/14463

(51) Int. Cl. A61K 31/715; A61K 38/38; A61P 17/00

(52) U.S. Cl. 514/12; 514/54

(57) ABSTRACT

A method of treating aging of skin including applying a therapeutically effective amount of an anti-aging dermatological care composition including at least one cosmetic formulation agent and oligogalacturonides having a degree of polymerization between 1 and 5 that stimulates adherence of basal keratinocytes to laminin V and/or collagen IV and reduces communication disturbances between a subject's dermis and epidermis and reduces diminishment in interkeratinocyte cohesion within the epidermis, to skin.

### RELATED APPLICATION

This application is a divisional of U.S. application Ser. No. 10/839,420, filed May 5, 2004, incorporated herein by reference, which is a continuation of International Application No. PCT/FR02/03844, with an international filing date of Nov. 8, 2002 (WO 03/039509, published May 15, 2003), which is based on French Patent Application No. 01/14463, filed Nov. 8, 2001.

### TECHNICAL FIELD

This disclosure relates to new cosmetic compositions for skin care with an anti-aging intent. More particularly, the technology herein pertains to the cosmetic use of a mixture of oligosaccharides of the type obtained by enzymatic hydrolysis of a pectin. The oligosaccharides stimulate adhesion of keratinocytes to proteins of the dermoepidermal junction (laminin V and collagen IV). The compositions can resolve disturbances in communication between the dermis and the epidermis and the decrease in the interkeratinocyte cohesion within the epidermis that appear during cutaneous aging and thereby restore epidermal cohesion.

### BACKGROUND

The basal membrane of the skin or dermoepidermal junction (DEJ) corresponds to the zone comprised anatomically between the basal cells of the epidermis and the more superficial layers of the dermis. This is a zone of adherence between the epidermis and the dermis, providing for the control of the filtration of small molecules and the maintenance of the adjacent cells (Damour O., M. C. Martini and P. Rousselle, October 1998, Cutaneous Aging, pub. Flash Media).

The DEJ comprises specific attachment complexes, the hemidesmosomes, whose function is to provide a bond between basal keratinocytes of the epidermis and the subjacent basal membrane (Kelly, 1966, J. Cell. Biol., 28: 51-73). The DEJ plays a very important role both on the mechanical level, since it enables solid anchoring of the epidermis, as well as on the biological level, since it intervenes in cell signalization via the integrin family of receptors.

Integrins are transmembranal glycoproteins located on the basal part of the keratinocyte in contact with the DEJ. They have an extracellular part enabling recognition with the characteristic proteins of the DEJ. Among the components of the DEJ, we can cite two proteins which play a fundamental role within the DEJ: laminin V, which is a constitutive protein of the hemidesmosomes, and collagen IV.

These proteins, via the membrane receptors (the α6β4 α3β1 integrins for laminin V and the α2β1 integrins for collagen IV), enable: adhesion of the basal keratinocytes to the support according to a clearly defined orientation, and transmission of signals from the dermis to the epidermis as proliferation signals, differentiation of the keratinocytes. These integrins are, thus, veritable zones of dialogue between the interior and the exterior of the cell and beyond that of the basal layer, they contribute, by promoting cellular adhesion to better communication between the principal compartments of the skin, the dermis and the epidermis.

More recently, it has been shown that adhesion of the basal keratinocytes to the major proteins of the DEJ, such as laminin V, collagen IV and fibronectin, appears to regulate the expression of the junctions (junction gap) between the keratinocytes of the epidermis (Lampe et al., J. Cell. Biol., 1998, 1735-1747). Thus, an increase in the interactions between especially laminin V and the basal keratinocytes via integrins such as α6β4 α3β1 generates the emission of signals within the epidermis to promote formation of intercellular junctions and enable better communication among keratinocytes of the epidermis.

During cutaneous aging, there is seen a flatting and a thinning of the DEJ. The adherence properties of the epidermis are decreased because of a diminishment in the expression of the integrins specifically involved in the adhesion of the basal keratinocytes (Levarlet et al., 1998, J. Invest. Dermatol., 3: 172-9). All of these changes lead to a diminishment in communication between the various compartments, probably contributing to dermoepidermal disorganization.

Even though not all of the mechanisms have been clarified, everything leads one to believe that an augmentation of the adherence of the cells, especially to the DEJ, enables reestablishment of better dermoepidermal communication as well as better epidermal cohesion leading to the restoration of a better coordination of the functions of the skin. In fact, disturbances in the dermoepidermal communication, on the one hand, and at the level of the cohesion among the keratinocytes of the epidermis, on the other hand, could lead to disturbances in coordination of the cell functions such as proliferation and/or epidermal differentiation.

It would, therefore, be advantageous to provide means enabling augmentation specifically of the adherence of basal keratinocytes to the two major proteins of the DEJ, which are laminin V and collagen IV, to resolve disturbances in communication between the dermis and the epidermis, and diminishment in interkeratinocyte cohesion that appear during cutaneous aging.

### SUMMARY

We provide dermatological compositions including a therapeutically effective amount of an agent stimulating adherence of basal keratinocytes to laminin V and/or collagen IV that reduces communication disturbances between a subject's dermis and epidermis and reduces diminishment in interkeratinocyte cohesion within the epidermis.

We also provide a method of preparing oligogalacturonides including hydrolysis of a pectin solution at a concentration of about 0.1 to about 10% at an acidic pH, by addition to the pectin solution of an enzyme solution of about 10 to about 1000 polygalacturonase units to obtain in a final solution of about 1 to about 10 pectinase units; stopping the hydrolysis; separation of resulting high-molecular-weight polymers from the final solution; and recovery of the oligogalacturonides.

We further provide a method of treating aging of skin including applying a therapeutically effective amount of the composition stimulating adherence of basal keratinocytes to laminin V and/or collagen IV that resolves communication disturbances between a subject's dermis and epidermis and diminishment in interkeratinocyte cohesion within the epidermis to the skin.

We still further provide a method of reducing decreases in interkeratinocyte cohesion is skin including applying a therapeutically effective amount of the composition stimulating adherence of basal keratinocytes to laminin V and/or collagen IV that resolves communication disturbances between a subject's dermis and epidermis and diminishment in interkeratinocyte cohesion within the epidermis to the skin.

### BRIEF DESCRIPTION OF THE DRAWINGS

FIGS. 1A and 1B are photographs showing the number of cells adhered to laminin V. FIG. 1A is a control and FIG. 1B is in accordance with aspects of the invention.

FIGS. 2A and 2B are photographs showing the number of cells adhered to collagen IV.

FIG. 2A is a control and FIG. 2B is in accordance with aspects of the invention.

### DETAILED DESCRIPTION

We provide cosmetic or dermatological compositions for skin care and, more particularly, for combating aging of the skin, comprising oligogalacturonides as an active ingredient. Application of the composition promotes adhesion of cells to two proteins of the DEJ by an enhanced availability of the α6β4 receptors for laminin V and the α2β1 receptors for collagen IV. The composition according to aspects of the invention induces activation of these receptors by means of a change of conformation.

The oligogalacturonides of the compositions preferably have a degree of polymerization between about 1 and about 5. The oligogalacturonides of the compositions are at least partially methylated or esterified. The cosmetic compositions comprise, in dry equivalent in relation to the total weight of the composition, from about 0.01 to about 5%, preferably about 0.5%, of oligogalacturonides.

In addition to the oligogalacturonides, the cosmetic compositions can also comprise other active substances, more specifically plant extracts. Examples of such extracts include: a yam extract (Dioscorea) with a content of diosgenin or pure diosgenin. This can be an extract containing about 15% of diosgenin or a solution of pure diosgenin; and pure or diluted beta carotene in the form of a suspension in oil, notably, a about 30% dilution.

The compositions can also comprise one or more formulation agents or additives of common and conventional use in cosmetic and dermatological compositions such as, as nonlimitative examples, softeners, colorants, film-forming agents, surface-active agents, perfumes, preservatives, emulsifiers, oils, glycols, sebum-absorbing agents, vitamins and the like. Those skilled in the art know which formulation agents to add to the compositions and what amounts in relation to the desired properties.

The compositions can be made available in any form known in art of cosmetology and dermatology without any pharmaceutical restriction other than application to the skin of the face or the body. The compositions are advantageously presented in the form of a gel, a cream, an emulsion, a milk, a spray and the like.

The oligogalacturonides of the compositions are advantageously obtained by enzymatic hydrolysis of a pectin. Pectin is constituted of a principal chain called “pectic acid” comprising a chain of galacturonic acid type sugars. The constitutive sugars can be methylated or esterified, and the proportion of transformed sugars is characteristic of a plant species. The principal chain is sometimes interrupted by the insertion of a side chain of neutral sugars such as rhamnose or glucose.

The oligogalacturonides, previously referred to as oligosaccharines, have been described as veritable plant hormones (Darvill et al., Glycobiology, vol. 2, no. 3, pp 181-198, 1992). They can be prepared by hydrolysis of pectin; the size or degree of polymerization of the oligogalacturonides is a function of the conditions of the hydrolysis reaction.

We have now developed a method for preparing oligogalacturonides providing an industrial product that is effective as a cosmetic agent. Numerous pectins are available commercially in large quantity and variable quality. These are often standardized pectins that frequently contain added sugars to enable homogenization of the viscosities among the batches. In fact, the first use of these pectins is linked to their gelling properties and as viscosity agents. The oligogalacturonides employed in the compositions of the invention are preferably prepared by hydrolysis of pectin having a low degree of methylation and esterification to be as close as possible to polygalacturonic acid.

The pectins of the type HERBSTREITH and FOX Classic AU 910 obtained from apples (pectin type HB AU) are an example of such a pectin. The enzymes used for the hydrolysis of pectin are of the industrial type frequently used in the fruit juice processing industry. These are cocktails of enzymes conceived to cut the membranal pectins and thereby enable better extraction of the fruit juices by making the structures fragile prior to pressing. They also make possible clarification of the juices in which turbidity—often linked to pectins—is not desirable. The enzyme marketed by the company LYVEN as Clarification granulated is an example of such an enzyme. The enzyme cocktail comprises pectinases having principally polygalacturonase, methyl esterase and polygalacturonase lyase activities.

A preferred method for preparing oligogalacturonides comprises the following steps:

hydrolysis of a pectin solution at a concentration of about 0.1 to about 10%, preferably at about 1%, at a pH of about 4.5, by addition to the pectin solution of an enzyme solution comprising from about 10 to about 1000, preferably about 100, polygalacturonase units to obtain in the final solution from about 1 to about 10, preferably about 4, pectinase units;

stopping the hydrolysis; and

separation of the high-molecular-weight polymers and recovery of the oligogalacturonides.

The hydrolysis is advantageously performed at about 50° C. for about 2 hours then stopped by heating at about 70° C. for about 1 hour or at about 100° C. for about 5 minutes. After cooling, the high-molecular-weight polymers are preferably precipitated by addition of HCl 1N then eliminated by centrifugation, e.g., at about 5000 g for about 30 minutes or by filtration. The pH of the supernatant is then readjusted to a value between about 6 and about 8, e.g., on the order of about 6.9.

We also provide for the use in cosmetics or for the preparation of a pharmaceutical composition, notably a dermatological composition, of oligogalacturonides as defined above as an agent stimulating adherence of the basal keratinocytes to the two major proteins of the DEJ, which are laminin V and collagen IV, and resolve the disturbances of communication between the dermis and the epidermis, and the diminishment in the interkeratinocyte cohesion within the epidermis, which appear during cutaneous aging.

We also provide a cosmetic method for resolving the disturbances in communication between the dermis and the epidermis, and the diminishment in interkeratinocyte cohesion within the epidermis, which appear during cutaneous aging and thereby to restore the epidermis cohesion comprising applying to the skin a therapeutically effective amount of oligogalacturonides or of a composition containing them as defined above.

Other advantages and characteristics will emerge from the examples below concerning the preparation of oligogalacturonides and their use as cosmetic agent.

### EXAMPLES

### I. Preparation of the Oligogalacturonides

1) Operating Procedure

Pectin: put in solution at from 0.1 to 10% (1% of pectin HB AU910).

The pH is adjusted to 4.5, preferably with an acetic acid solution.

Enzyme: A solution is prepared corresponding to 10 to 1000, preferably 100, polygalacturonase units. The enzymatic solution is added to the pectin solution to obtain at the end a solution of 1 to 10, preferably 4, pectinase units. Hydrolysis is performed at 50° C. for 2 hours then stopped by heating at 70° C. for 1 hour or at 100° C. for 5 minutes. After cooling, the high-molecular-weight polymers are precipitated by addition of HCl 1N then eliminated by centrifugation, e.g., at 5000 g for 30 minutes or by filtration. The pH of the supernatant is readjusted at the end to a value comprised between 6 and 8, e.g., on the order of 6.9.

The oligogalacturonides formed are advantageously atomized or lyophilized at the end to enable better preservation and easier handling.

2) HPLC Analysis of the Oligogalacturonides Formed

An analysis technique producing a chromatographic profile of the oligogalacturonides formed was developed.

Column: TSK gel DEAE 5-PW (TOSOHAAS)

Eluent: CH₃COONH₄ 1M/H₂O in elution gradient

Mobile flow rate: 1 ml/min

Light diffusion evaporative detector (DEDL; ALTECH).

Oven Temperature: 130° C.

Nitrogen flow rate: 4.0 SLPM (standard liter per minute): standard conditions for H₂O as solvent.

The selection of the gradient is presented in Table 1 below:

TABLE 1

| Time in minutes | % A (CH₃COONH₄ 1M) | % B (H₂O) |
|---|---|---|
| 0 | 10 | 90 |
| 1 | 30 | 70 |
| 3 | 30 | 70 |
| 30 | 50 | 50 |
| 45 | 50 | 50 |

We observe a distribution of the oligomers by size (dp: degree of polymerization) from 1 to 5.

The of a standard does not allow quantitative determination of the oligomers of dp 4 and 5, but quantitative determination of the shorter oligomers is possible.

If we proceed according to the conditions described for hydrolysis of a pectin solution of concentration 10 g/l, the total concentration in mono-, di- and trigalacturonic acid is approximately 4.5 g/l at the end, thus approximately 45% by weight of the pectin put in solution.

### II. Effects of the Oligogalacturonides on the Adhesion of Human Keratinocytes to Laminin V and to Collagen IV

1) Material and Methods

a) Culture of the Keratinocytes in Defined Medium

The culture medium employed was the defined medium for keratinocyte culture 154 (+additive HKGS) manufactured by Cascade Inc. (USA) and marketed by Tébu (France) containing 0.2 mM of CaCl₂, pH 7.2 to 7.4.

The keratinocytes were obtained according to the technique described by Boyce and Ham (Boyce S T, Ham R G, J. Invest. Dermatol., 1983, 81, 33s-40s). Pieces of human skin obtain from human prepuces (circumcision) were treated in a manner to isolate their basal human keratinocytes. 3·10⁴ live cells were then seeded per cm² on 25-cm² tissue culture dishes (Corning, Polylabo, France).

The keratinocytes were cultured at 37° C. in an incubator with CO₂ (5% of CO₂, 95% of air and 98% humidity). The medium was changed every two days. Subculture took place when the cells reached subconfluence. The cell layer was then rinsed with PBS, then the cells were trypsinated using the conventional trypsination technique (Trypsin-EDTA (0.05-0.02%) at 37° C.). The cells were then seeded in 75-cm² culture dishes.

Freezing the cells, 3 to 5 million per ampoule, was performed in the culture medium employed, in the presence of 10% dimethyl sulfoxide (DMSO) and 20% of calf serum in a volume of 1 ml.

b) Quantitative Analysis of Cellular Adherence by a Calorimetric Test

Preparation of the Adherence Substrates

A dose-response for each of the adhesion substrates was determined to establish the ideal concentration of the adherence proteins that will subsequently be used.

Collagen IV (Becton Dickinson, France), fibronectin (Becton Dickinson, France) and laminin 5, purified in the laboratory (Pousselle P. et al., J. Cell. Biol., 1991, 114(3); 567-576) were used in our experiments. A range of seven decreasing concentrations was created by successive dilution in distilled water, from a starting solution of 10 μg/ml. These solutions were immediately distributed on 96-well culture plates (Costar, Dutscher, Brumath, France) at the rate of 100 μg per well. The plates were then placed at +4° C. for 16 to 18 hours. The solutions were then removed by turning over the plates and each well was saturated by an aqueous solution of SAB 1% (3 supplementary wells without substrate were subjected to the same treatment and functioned as blanks).

Test of Cellular Adherence

The cells were trypsinated as described above, then suspended in the medium 154 without additives (3×10⁵ cells/ml) then seeded in passage 2 in multiwell plates of 100 μl/well.

Evaluation of the Cellular Adherence Test

After seeding of the cells, the multiwell plates were placed in an incubator at 37° C. for a duration of 45 minutes in the presence of or absence of the oligogalacturonide mixture at different noncytotoxic concentrations in the culture medium. A positive control was implemented in parallel (manganese chloride (0.5 mM)) to validate the experiment. After incubation, the cells were observed with a phase-contrast microscope to verify that the test had taken place correctly.

The characteristic spread of the keratinocytes on laminin 5 (Rousselle P. and Aumilley M., J. Cell. Biol., 1994, 125(1): 205-214) was taken into account. After rinsing, the remaining cells, adherent to the substrate, were fixed with a 1% glutaraldehyde solution in PBS for 15 minutes. After elimination of the fixative, the cells were stained with a crystal violet solution diluted to 1% in distilled water for 30 minutes. After intensive rinsing with water, the cells were permeabilized with a 0.02% triton solution for 15 minutes to solubilize the crystal violet.

An absorbance reading was performed at 570 nm using an ELISA plate reader. Each experimental point was performed in three samples. The blank value represents the mean of the absorbance of 3 control wells (BSA). This value was subtracted from each of the optical density values obtained for the experimental points. We then calculated the means of the three absorbance values for each of the triplicates.

2) Results

a) Study of the Adhesion of Normal Human Keratinocytes on Laminin V

Manganese chloride (0.5 mM) was used as a positive control for the adhesion of the cells to the substrate. The adherence result obtained with the cells without active ingredient and without positive control was set arbitrarily at 100%. The active ingredient identified here constitutes the reference solution of oligosaccharides type oligo G04 (not lyophilized).

The results obtained are presented in Table 2 below.

TABLE 2

| Laminin 5 | | | Laminin 5 | | |
|---|---|---|---|---|---|
| 3 μg/ml | % | SD | 1.5 μg/ml | % | SD |
| Cells alone | 100 | | Cells alone | 100 | |
| MnCl₂ | 97 | 6.4 | MnCl₂ | 109 | 5.1 |
| Solvent | 101 | 0.96 | Solvent | 107 | 7.9 |
| Active 5% | 103 | 0.16 | Active 5% | 119 | 1.4 |
| 1% | 116 | 14 | 1% | 100 | 7.5 |
| 0.01% | 105 | 7.2 | 0.01% | 105 | 7.5 |

The mixture induced an augmentation of 116% on laminin V at the concentration of 1%.

b) Study of the Adhesion of Normal Human Keratinocytes on Collagen IV

Manganese chloride (0.5 mM) was used as positive control for the adhesion of the cells to the substrate. The adherence results obtained with the cells without active ingredient and without positive control was set arbitrarily at 100%.

The results obtained are presented in Table 3 below.

TABLE 3

| Collagen IV | | | Collagen IV | | |
|---|---|---|---|---|---|
| 20 μg/ml | % | SD | 10 μg/ml | % | SD |
| Cells alone | 100 | | Cells alone | 100 | |
| MnCl₂ | 357 | 29 | MnCl₂ | 280 | 10 |
| Solvent | 105 | 2.3 | Solvent | 90 | 3 |
| Active 5% | 84 | 4.7 | Active 5% | 66 | 6 |
| 1% | 126 | 17 | 1% | 90 | 15 |
| 0.01% | 101 | 5.9 | 0.01% | 80 | 2.5 |

The mixture induced an augmentation of 126% on collagen IV at the concentration of 1%.

c) Study of the Morphology of Normal Human Keratinocytes after Adhesion on Laminin V or Collagen IV

Visualization of the spread and form of the adhered cells was performed by a demonstration of the cytoskeleton of actin by performing immunolabeling with phalloidin coupled to FITC.

Adhesion to Laminin V

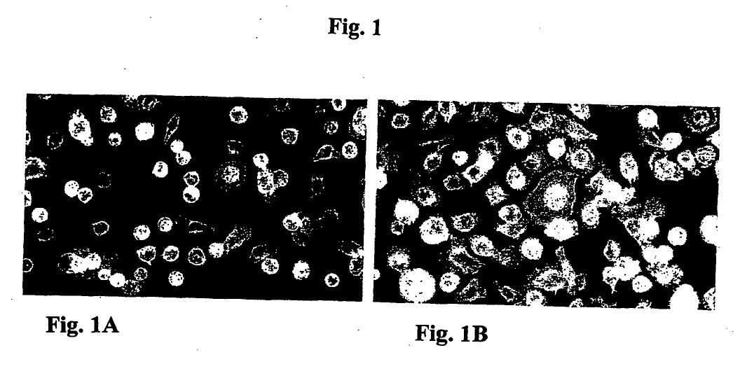

FIGS. 1A and 1B show that, in the presence of the active ingredient used at 0.01% (FIG. 1B), the number of cells having adhered to laminin V was greater than in the control (FIG. 1A). These cells are more spread apart and the representative actin network of the cellular cytoskeleton is much better organized. These morphological changes due to the presence of the active ingredient (compared to the control) indicate that this ingredient truly promotes the recruitment and activation of the integrin α6β4.

Adhesion to Collagen IV

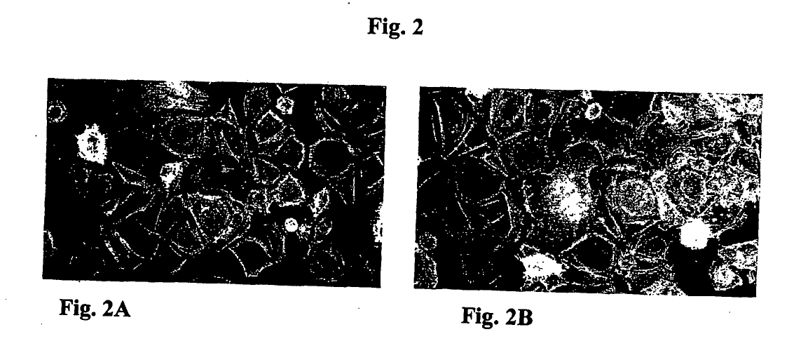

In the absence (FIG. 2A) or in the presence of the active ingredient (FIG. 2B), the cells adhere to collagen IV. However, it can be seen that in the presence of the active ingredient (FIG. 2B), the cells are larger, with a rather rounded shape with a very good cortical organization of the actin. In this case, the cells are joined, pressed against each other, indicating that the active ingredient stimulates cell-cell contacts and intercellular cohesion.

In conclusion, augmentation of the adherence of cells to laminin V in the presence of the active ingredient appears to come about via an augmentation of the expression of α6β4. The morphology and the spread of the cells on collagen IV in the presence of the active ingredient indicates that there is produced a recruitment of integrins α2β1 (specific to collagen IV) and that there also exists an enhanced cohesion among the keratinocytes.

### III. Incorporation in a Cosmetic Formulation

In dry equivalents, the oligogalacturonides can be incorporated at the rate of about 0.1 to about 5%, preferably at the rate of about 0.5%.

1) Example of Composition in Emulsion Form

| Water | QSP |
|---|---|
| Oligogalacturonides dp 1 to 5 (dry matter) | 0.01 to 5% |
| Mixture of preservatives | 1.5% |
| Propylene glycol | 5.00% |
| Xanthan gum | 0.30% |
| Acrylic/acrylate copolymer | 0.50% |
| Stearic acid 100 OE** | 3.00% |
| Sorbitan stearate | 2.00% |
| Sorbitan laurate 20 OE | 3.00% |
| Cetyl stearic alcohol | 1.50% |
| Bee's wax | 1.00% |
| Wheat germ oil | 5.00% |
| Dimethicone | 2.00% |
| Cyclomethicone | 5.00% |
| Polyacrylamide gel | 2.00% |
| Perfume | 0.10% |

**stearic acid with 100 moles of OE

2) Example of Composition in Cream Form

| Water | QSP |
|---|---|
| Oligogalacturonides dp 1 to 5 (dry matter) | 0.001% to 0.1% |
| Xanthan gum | 0.30% |
| Sequestration agent (e.g., EDTA) | 0.05% |
| Preservatives | 1.50% |
| Acid C18 | 2.50% |
| Acid C16 | 2.50% |
| Trilaurin | 1.00% |
| Shea butter | 3.00% |
| Tocopherol acetate | 0.05% |
| β-bisabolol | 0.05% |
| Vegetable oil (wheat germ) | 5.00% |
| Dimethicone | 3.00% |
| Polyacrylic acid | 0.30% |
| TEA (triethanolamine) | 1.50% |
| Perfume | 0.10% |

1. A method of treating aging of skin comprising:
applying a therapeutically effective amount of an anti-aging dermatological care composition comprising at least one cosmetic formulation agent and oligogalacturonides having a degree of polymerization between 1 and 5 that stimulates adherence of basal keratinocytes to laminin V and/or collagen IV and reduces communication disturbances between a subject's dermis and epidermis and reduces diminishment in interkeratinocyte cohesion within the epidermis, to skin.

2. The method according to claim 1, wherein the oligogalacturonides are obtained by enzymatic hydrolysis of a pectin, the hydrolysis being performed by an enzyme cocktail comprising pectinases having polygalacturonase, methyl esterase and polygalacturonase lyase activities.

3. The method according to claim 1, wherein the oligogalacturonides enhance availability of α6β4 receptors for laminin V and α2β1 receptors for collagen IV.

4. The method according to claim 1, wherein the oligogalacturonides are at least partially methylated or esterified.

5. The method according to claim 1, wherein the oligogalacturonides comprise about 0.01% to about 5% by weight of the composition.

6. The method according to claim 1, wherein the composition further comprises at least one plant extract as another active component.

7. The method according to claim 6, wherein the plant extract is selected from the group consisting of yam and beta carotene.
